(19) United States
(12) Patent Application Publication
(10) Pub. No.: US 2011/0027372 A1
(43) Pub. Date: Feb. 3, 2011

(54) MULTIPARTICULATES COMPRISING LOW-SOLUBILITY DRUGS AND CARRIERS THAT RESULT IN RAPID DRUG RELEASE

(76) Inventors: Leah Elizabeth Appel, Bend, OR (US); Dwayne Thomas Friesen, Bend, OR (US); David Keith Lyon, Bend, OR (US); Scott Baldwin McCray, Bend, OR (US); Joshua Richard Shockey, Bend, OR (US)

Correspondence Address: CHERNOFF, VILHAUER, MCCLUNG & STENZEL, LLP, 601 SW Second Avenue, Suite 1600, PORTLAND, OR, 97204-3157, US

(21) Appl. No.: 12/902,033

(22) Filed: Oct. 11, 2010

Related U.S. Application Data
(62) Division of application No. 11/920,468, filed on Nov. 14, 2007.
(60) Provisional application No. 60/688,511, filed on Jun. 7, 2005.

(51) Int. Cl. A61K 9/14

(52) U.S. Cl. 424/489

(57) ABSTRACT

Multiparticulates of low-solubility drugs and carriers that result in rapid release of the drug are disclosed.

### BACKGROUND OF THE INVENTION

Multiparticulates are well-known dosage forms that comprise a multiplicity of particles whose totality represents the intended therapeutically useful dose of a drug. When taken orally, multiparticulates generally disperse freely in the gastrointestinal tract, exit relatively rapidly and reproducibly from the stomach, maximize absorption, and minimize side effects. See, for example, Multiparticulate Oral Drug Delivery (Marcel Dekker, 1994), and Pharmaceutical Pelletization Technology (Marcel Dekker, 1989).

The preparation of drug-containing multiparticulates by melting a matrix material, mixing drug with the melt, forming the drug-containing melt into droplets and cooling the droplets is known. Such processes are generally referred to as “melt-congeal” processes. However, such melt-congeal processes use relatively hydrophobic, waxy materials for the matrix. When low-solubility drugs are incorporated into such a waxy matrix and formed into multiparticulates that are introduced to an aqueous environment of use, drug release is not immediate, but rather is by way of diffusion through, for example, liquid-filled pores of the matrix. However, the rate of drug release from such multiparticulates is limited by their degree of porosity. There is therefore a need for multiparticulates that are capable of quickly releasing low-solubility drugs.

### BRIEF SUMMARY OF THE INVENTION

The present invention overcomes the drawbacks of the prior art by providing multiparticulates comprising a low-solubility drug and a highly water-soluble matrix or a pore-forming carrier that are capable of rapidly releasing the low-solubility drug into an aqueous environment of use.

According to the present invention it has been found that sugar alcohols with a high ratio of melting temperature (Tm) to glass-transition temperature (Tg) that are extremely water-soluble may be used to formulate low-solubility drug-containing multiparticulates that rapidly release the drug into an aqueous environment of use.

Thus, in one aspect, the invention provides a pharmaceutical composition comprising multiparticulates of a low-solubility drug and a sugar alcohol, wherein the multiparticulates have volume-weighted mean diameter of from about 10 μm to about 1000 μm, and wherein the sugar alcohol makes up at least 30 wt % of the multiparticulates and the sugar alcohol having a Tm to Tg ratio in degrees Kelvin (K) of at least about 1.5.

In another aspect, the invention provides a process for forming multiparticulates comprising the steps of (a) forming a feed mixture comprising a low-solubility drug, a sugar alcohol, and water; (b) forming droplets of the feed mixture; and (c) solidifying the droplets to form multiparticulates.

A chief advantage of this embodiment of the present invention is the provision of drug-containing multiparticulates capable of rapidly disintegrating in an aqueous environment, thereby rapidly releasing the drug.

It has also been found that certain carriers and mixtures thereof may be used to formulate low-solubility drug-containing multiparticulates that become highly porous upon introduction to an aqueous environment, thereby allowing rapid ingress and egress of water into and out of the multiparticulates, which in turn speeds up the diffusion of drug therefrom into an aqueous environment of use.

Thus, in another aspect, the invention provides a pharmaceutical composition comprising multiparticulates of a low-solubility drug and a pore-forming carrier; the pore-forming carrier is selected from (i) a mixture of glyceryl dibehenate and polyethylene glycol behenate, (ii) an anionic emulsifying wax, and (iii) mixtures thereof.

In yet another aspect, the invention provides a process for forming multiparticulates comprising the steps of (a) forming a molten mixture comprising the low-solubility drug and a pore-forming carrier; (b) forming droplets of the molten mixture; and (c) solidifying the droplets to form the multiparticulates.

A chief advantage of this embodiment of the invention is the provision of drug-containing multiparticulates capable of providing rapid release of a low-solubility drug from multiparticulates exposed to an aqueous environment.

Finally, the multiparticulates of the invention typically are smooth, spherical particles, which impart better flow characteristics to the multiparticulates that in turn facilitate processing the multiparticulates.

### BRIEF DESCRIPTION OF THE DRAWINGS

FIG. 1 is a Scanning Electron Microscope (SEM) photo of the multiparticulates of Example 1.

FIG. 2 is an SEM photo of the inside of one of the multiparticulates of Example 1.

FIG. 3 is a light-microscope photo of multiparticulates of Example 1 prior to exposure to water.

FIG. 4 is a light-microscope photo of multiparticulates of Example 1 about 5 seconds after exposure to deionized water.

FIG. 5 is an SEM photo of the multiparticulates of Example 2.

FIG. 6 is a light-microscope photo of multiparticulates of Example 2 prior to exposure to water.

FIG. 7 is a light-microscope photo of multiparticulates of Example 2 about 5 seconds after exposure to deionized water.

FIG. 8 is a graph of the in vitro drug release rate of the multiparticulates of Example 4, as compared to multiparticulates of the same drug and another, more hydrophobic carrier.

FIG. 9 is a Scanning Electron Microscope (SEM) photo of exemplary drug-loaded multiparticulates of the invention.

FIG. 10 is an SEM photo of the multiparticulates of FIG. 9 after exposure to water for one minute at room temperature.

### DETAILED DESCRIPTION OF PREFERRED EMBODIMENTS

As used in the specification and claims, the term “about” means the specified value±10% of the specified value.

The compositions of the present invention comprise a plurality of “multiparticulates.” The term “multiparticulate” is intended to embrace a dosage form comprising a multiplicity of particles whose totality represents the intended therapeutically useful dose of drug. While the multiparticulates can have any shape and texture, it is preferred that they be generally spherical, with a relatively smooth surface texture. This is in contrast to granules made by granulation processes that consist of agglomerates of small particles in which the original particles can still be identified. The physical characteristics of multiparticulates generally lead to excellent flow properties, improved “mouth feel,” ease of swallowing and ease of uniform coating, if desired.

The multiparticulates of the invention comprise a sugar alcohol or a pore-forming carrier. The sugar alcohol and/or pore-forming carrier, referred to herein as a matrix material, serves two functions. First, the matrix material binds the drug particles together. Second, the matrix material allows the multiparticulate to be formed into a relatively smooth, round sphere. As detailed herein below, the multiparticulates of the present invention are essentially drug particles and optionally other excipients, encapsulated within a continuous phase of matrix material. Because of this, a sufficient amount of matrix material must be present so as to encapsulate the drug to form smooth and spherical multiparticulates.

A useful measurement in describing small particles is volume-weighted mean diameter. The volume-weighted mean diameter assumes a gaussian size distribution, with approximately 85% of the particle volume being within about 30% of the reported diameter. The multiparticulates generally are of a volume-weighted mean diameter of from about 10 μm to about 1000 μm, preferably from about 30 μm to about 300 μm. Multiparticulates are preferred because they are amenable to use in scaling dosage forms according to the weight of an individual patient in need of treatment by simply scaling the mass of particles in the dosage form to comport with the patient's weight. They are further advantageous since they allow the incorporation of a large quantity of drug into a simple dosage form such as a sachet that can be formulated into a slurry that can easily be administered orally. Multiparticulates also have numerous therapeutic advantages over other dosage forms, especially when taken orally, including (1) improved dispersal in the gastrointestinal (GI) tract, (2) more uniform GI tract transit time, and (3) reduced inter- and intra-patient variability.

The multiparticulates formed by the process of the present invention are designed for rapid release of drug after introduction to a use environment. As used herein, a “use environment” can be either the in vivo environment of the GI tract of a mammal, particularly a human, or the in vitro environment of a test solution. Exemplary test solutions include aqueous solutions at 37° C. comprising (1) 50 mM KH₂PO₄ and 0.5 wt % of the surfactant polyoxyethylene 30 oleate (commercially available as TWEEN 80 from ICI Americas, Inc. of Wilmington, Del.) at pH 7.3; and (2) 50 nM NaH₂PO₄, at pH 6.5, containing 2 wt % sodium lauryl sulfate (SLS). It has been determined that in vitro dissolution tests in such solutions provide a good indicator of in vivo performance and bioavailability.

Further details regarding the multiparticulates of the invention, including suitable low-solubility drugs to be administered by them, suitable sugar alcohols and pore-forming carriers for forming them, processes for forming them, and suitable dosage forms containing the multiparticulates are set forth in more detail below.

### Low-Solubility Drugs

The multiparticulates of the present invention are particularly suitable for the rapid delivery of low-solubility drugs. As used herein, “low-solubility drug” means a drug having an aqueous solubility of less than about 10 mg/mL at pH 6-7. The drug may have an even lower aqueous solubility, such as less than about 5 mg/mL, less than about 1 mg/mL, less than about 0.5 mg/mL, less than about 0.1 mg/mL, and even less than about 0.05 mg/mL. A useful measure of the solubility of low-solubility drugs is a dose-to-aqueous solubility ratio, which may be calculated by dividing the dose (in mg) by the aqueous solubility (in mg/mL). In general, it may be said that drugs useful in the invention have a dose-to-aqueous solubility ratio greater than about 10 mL, and more typically greater than about 100 mL, where the aqueous solubility in mg/mL is the minimum value observed in an aqueous solution at pH 6-7, and the dose is in mg.

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, cholesterol-reducing agents, triglyceride-reducing agents, anti-atherosclerotic agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, and cholesteryl ester transfer protein inhibitors.

Each named drug should be understood to include any pharmaceutically acceptable forms of the drug. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs. Specific examples of low-solubility drugs suitable for use in the present invention include acyclovir, amlodipine, apomorphine, atorvastatin, celecoxib, chlorthalidone, clarithromycin, digitoxin, digoxin, erythromycin, famotidine, fluconazole, glipizide, griseofulvin, lidocaine, nadolol, nelfinavir, nifedipine, paroxetine, phenobarbital, prednisolone, sertraline, sildenafil, spironolactone, testosterone, thiabendazole, torcetrapib, valdecoxib, voriconazole, and ziprasidone.

The low-solubility drug makes up at least about 5 wt % of the total weight of the multiparticulate. Preferably, the low-solubility drug makes up at least about 10 wt % of the total weight of the multiparticulate, more preferably at least about 15 wt %, even more preferably at least about 20 wt %, still more preferably at least about 25 wt %, and most preferably at least about 30 wt %. However, excessive drug loading of the multiparticulates can lead to difficulties during their processing. Therefore, it is preferred that the low-solubility drug make up less than about 80 wt % of the total weight of the multiparticulate, more preferably less than about 70 wt %. Thus, the low-solubility drug will generally constitute from at least about 5 wt % to less than about 80 wt % of the total weight of the multiparticulate. Preferably, the low-solubility drug constitutes from at least about 10 wt % to less than about 70 wt % of the total weight of the multiparticulates.

The drug present in the multiparticulate can be amorphous or crystalline. By “amorphous” is meant that the compound is not “crystalline.” By “crystalline” is meant that the compound exhibits long-range order in three dimensions. The amount of crystalline and amorphous drug present in the multiparticulates may be characterized by techniques known in the art such as powder x-ray diffraction (PXRD) crystallography, solid state NMR, or thermal techniques such as differential scanning calorimetry (DSC). Preferably, at least 70 wt % of the drug is crystalline, more preferably, at least 80 wt % of the drug is crystalline, even more preferably at least 90 wt % of the drug is crystalline, and most preferably at least 95 wt % of the drug in the multiparticulate is crystalline.

### Multiparticulates Comprising Sugar Alcohols

In one aspect, the multiparticulates of the invention comprise a sugar alcohol. Sugar alcohols, sometimes referred to in the art as polyols, contain carbon chains, with the carbon atoms generally bearing a hydroxyl group. Sugar alcohols generally are highly soluble in water at 25° C., preferably to at least about 1 wt %, more preferably to at least about 5 wt %, and most preferably to at least about 10 wt %.

The sugar alcohol makes up at least about 30 wt % of the total weight of the multiparticulates. Preferably, even higher amounts of sugar alcohol are present in the multiparticulates. Thus, the sugar alcohol makes up at least about 40 wt % of the total weight of the multiparticulate, more preferably at least about 50 wt %, and most preferably at least about 60 wt % of the total weight of the multiparticulate.

In another embodiment, the low-solubility drug and the sugar alcohol constitute at least 60 wt % of the total mass of the multiparticulate. Preferably, the drug and sugar alcohol constitutes at least about 70 wt %, and most preferably at least about 80 wt % of the total mass of the multiparticulate. In one embodiment, the multiparticulates consist essentially of a low-solubility drug and a sugar alcohol. Generally, the multiparticulates should not contain excipients that interfere with the rapid disintegration behavior of the multiparticulates.

In one embodiment, the low-solubility drug is in the form of particles distributed throughout the sugar alcohol. In this embodiment the multiparticulate comprises a plurality of drug-containing particles that are encapsulated within a continuous phase comprising the sugar alcohol.

The inventors found that when multiparticulates are made using sugar alcohols that do not rapidly crystallize during the multiparticulate-formation process, recovery of the multiparticulates can be problematic. The inventors addressed this problem by using a sugar alcohol that rapidly crystallizes, resulting in improved recovery and efficiency during the multiparticulate-formation process. Without wishing to be bound by any particular theory, it is believed that the tendency for a sugar alcohol to crystallize from an aqueous solution is characterized by the ratio of the Tm of the sugar alcohol (in K) to the glass-transition temperature for the alcohol, Tg (in K) of the sugar alcohol. The driving force for crystallization is controlled primarily by Tm, while the kinetic barrier to crystallization is controlled primarily by the Tg. The ratio, Tm/Tg (in K/K), indicates the relative propensity for a sugar alcohol to crystallize.

The inventors have discovered that sugar alcohols for which the ratio of Tm in K to Tg in K is at least about 1.5 can be effectively used to make multiparticulates with the desired properties. Preferably, this Tm/Tg ratio (K/K) is at least about 1.6, and more preferably at least about 1.65. Examples of suitable sugar alcohols include mannitol (Tm=438 K; Tg=234 K; Tm/Tg=1.87) and erythritol (Tm=394 K; Tg=231 K; Tm/Tg=1.71). Mixtures of such sugar alcohols may also be used.

Multiparticulates comprising a low-solubility drug and a sugar alcohol may be made by a process comprising the steps of (a) forming a feed mixture comprising the drug, the sugar alcohol, and water; (b) forming droplets of the feed mixture; and (c) solidifying the droplets to form the multiparticulates.

In one preferred embodiment, the multiparticulates are made by a process comprising the steps of (a) forming an aqueous sugar alcohol solution; (b) adding a low-solubility drug to the aqueous sugar alcohol solution to form a feed mixture; (c) forming droplets of the feed mixture; and (d) solidifying the droplets to form the multiparticulates.

The feed mixture should contain a sufficient amount of water and be at a sufficiently high temperature that (1) the sugar alcohol is dissolved in the feed mixture, and (2) the feed mixture is sufficiently fluid that the solution may be formed into droplets or atomized. Atomization of the mixture of low-solubility drug and the aqueous sugar alcohol solution may be carried out using any of the atomization methods described below. Generally, the feed mixture may be said to be sufficiently fluid that it will flow when subjected to one or more forces such as pressure, shear, and centrifugal force, such as that exerted by a centrifugal or spinning-disk atomizer. Generally, a feed mixture is sufficiently fluid for atomization when the viscosity of the feed mixture is less than about 20,000 cp, preferably less than about 15,000 cp, more preferably less than about 10,000 cp, even more preferably less than about 5,000 cp, and most preferably less than about 1,000 cp. The feed mixture is typically a suspension of solid drug particles in the aqueous sugar alcohol solution.

Virtually any process may be used to form the feed mixture. One method involves adding the sugar alcohol to water and then heating and stirring until it dissolves and the solution becomes sufficiently fluid as noted above, and then adding the drug to the solution. Alternatively, the sugar alcohol and low-solubility drug can be added to water and then the mixture heated and stirred until the sugar alcohol dissolves. It is understood that a small amount of the low-solubility drug will dissolve in the aqueous solution. A sufficient amount of low-solubility drug is used in the formulation that less than about 20 wt % of the drug is dissolved in the feed mixture, (meaning that about 80 wt % of the drug is undissolved and is suspended in the feed mixture). Preferably even smaller amounts of the drug are dissolved in the feed mixture. Thus, preferably less than about 15 wt % of the drug is dissolved in the feed mixture, more preferably less than 10 wt %, and most preferably less than about 5 wt %.

The sugar alcohol solution should contain sufficient water to dissolve the sugar alcohol. However, if too much water is included in the sugar alcohol solution, the time to solidify the multiparticulates increases, making processing difficult. Therefore, it is preferred that the sugar alcohol solution contain at least about 40 wt % sugar alcohol (60 wt % water), more preferably at least about 45 wt %, and more preferably at least 50 wt % sugar alcohol.

Generally, the aqueous sugar alcohol solution is heated to a temperature of at least about 75° C., preferably at least about 80° C., and most preferably at least about 85° C. These temperatures generally result in aqueous sugar alcohol solutions that are sufficiently fluid for atomization. Higher temperatures may be used, and are often advantageous for decreasing the time required to dissolve the sugar alcohol.

However, when the low-solubility drug is added to the sugar alcohol solution to form the feed mixture, high temperatures should be avoided, especially when the low-solubility drug is temperature-sensitive. Thus, it is preferred that when the low-solubility drug is added to the aqueous sugar alcohol solution the temperature be no greater than about 150° C., more preferably no greater than about 130° C., and most preferably no greater than about 120° C.

Once the feed mixture has been formed, it is formed into multiparticulates using the spray-congeal process described herein below.

Multiparticulates of the present invention comprising a low-solubility drug and a sugar alcohol rapidly disintegrate when introduced to an aqueous environment of use, thus providing rapid release of the low-solubility drug. Tests with multiparticulates of the invention have shown them to disintegrate rapidly upon exposure to water, e.g., within 5 minutes.

Specifically, following administration to a phosphate-buffered solution consisting of an aqueous solution of either (1) 50 mM KH₂PO₄ and 0.5 wt % of the surfactant polyoxyethylene 30 oleate (commercially available as TWEEN 80 from ICI Americas, Inc. of Wilmington, Del.) at pH 7.3, or (2) 50 mM NaH₂PO₄, at pH 6.5, containing 2 wt % sodium lauryl sulfate (SLS), drug-loaded multiparticulates of the invention completely disintegrate in about 5 minutes or less, and quite frequently in about 3 minutes or less.

Generally, following administration of the multiparticulates to the use environment, at least about 80 wt % of the low-solubility drug in the multiparticulates is released to the use environment within about 5 minutes, more preferably within about 3 minutes. The amount of drug released from the multiparticulates may be determined by introducing the multiparticulates to such a buffer solution and determining the concentration of drug dissolved in the buffer solution over time. However, because the drug has a low solubility, this test must be performed under conditions where there is a sufficient quantity of buffer so that all of the drug in the multiparticulates can be dissolved. Alternatively, a sample of the multiparticulates can periodically be removed from the buffer solution and the amount of drug remaining in the multiparticulates measured using standard techniques.

### Multiparticulates Comprising Pore-Forming Carriers

In another aspect, the multiparticulates of the invention comprise a pore-forming carrier. The pore-forming carrier preferably makes up at least about 20 wt % of the total weight of the multiparticulate. Preferably, the pore-forming carrier makes up at least about 30 wt % of the total weight of the multiparticulate, more preferably at least about 35 wt %, and most preferably at least about 40 wt %. However, it is also desirable that the multiparticulates contain a high percentage of drug. Thus, it is preferred that the pore-forming carrier make up less than about 95 wt % of the total weight of the multiparticulate, more preferably less than about 90 wt %, and most preferably less than about 80 wt %.

In one embodiment, the pore-forming carrier together with the low-solubility drug constitutes at least about 50 wt % of the total mass of the multiparticulate. Preferably, the carrier and drug constitutes at least about 60 wt %, more preferably at least about 70 wt %, and most preferably at least about 80 wt % of the total mass of the multiparticulate.

The pore-forming carrier is selected such that the multiparticulate forms a highly porous matrix upon exposure to an aqueous environment of use. Pore-forming carriers include (i) a mixture of glyceryl dibehenate and polyethylene glycol behenate, (ii) an anionic emulsifying wax and (iii) mixtures thereof. A preferred form of pore-forming carrier (i) comprises approximately equal amounts of glyceryl dibehenate and polyethylene glycol behenate, commercially available as COMPRITOL HD5 from Gattefosse Corporation of Westwood, N.J. An “anionic emulsifying wax” contains cetostearyl alcohol, purified water, and either sodium lauryl sulfate or a sodium salt of a similar sulfated higher primary aliphatic alcohol.

Preferably, the anionic emulsifying wax contains at least about 85 wt % cetostearyl alcohol, at least about 8 wt % sodium lauryl sulfate, and the balance purified water. A preferred form of anionic emulsifying wax comprises a mixture of cetostearyl alcohol, water and sodium lauryl sulfate, commercially available as LANETTE SX from Cognis Corporation of Cincinnati, Ohio.

In this aspect of the invention, the multiparticulates may optionally include dissolution-enhancing and viscosity-adjusting excipients to aid in forming the multiparticulates and to enhance the release rate of the low-solubility drug from the multiparticulates.

In one embodiment, an optional excipient is a dissolution enhancer. Dissolution enhancers increase the rate of dissolution of the drug from the multiparticulate. In general, dissolution enhancers are amphiphilic compounds and are generally more hydrophilic than the pore-forming carrier. When present, dissolution enhancers will generally make up about 0.1 to about 30 wt % of the total mass of the multiparticulate. Exemplary dissolution enhancers include alcohols such as stearyl alcohol, cetyl alcohol, and polyethylene glycol; surfactants, such as poloxamers (such as poloxamer 188, poloxamer 237, poloxamer 338, and poloxamer 407), docusate salts, polyoxyethylene alkyl ethers, polyoxyethylene castor oil derivatives, polysorbates, polyoxyethylene alkyl esters, sodium lauryl sulfate, and sorbitan monoesters; mixtures of mono-, di- and trialkyl glycerides and di-fatty acid esters of polyethylene glycol, commercially available as GELUCIRE 50/13 from Gattefosse Corporation; sugars such as glucose, sucrose, xylitol, sorbitol, and maltitol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; and mixtures thereof. Preferably, the dissolution enhancer is at least one surfactant. In one embodiment, the dissolution enhancer is selected from the group consisting of poloxamers, mixtures of mono-, di- and trialkyl glycerides and di-fatty acid esters of polyethylene glycol, and mixtures thereof.

Another useful class of optional excipients includes materials that adjust the viscosity of the molten mixture used to form the multiparticulates. Such viscosity-adjusting excipients will generally make up 0 to 25 wt % of the multiparticulate, based on the total mass of the multiparticulate. Examples of viscosity-reducing excipients include stearyl alcohol, cetyl alcohol, low molecular weight polyethylene glycol (e.g., less than about 1000 daltons), isopropyl alcohol, and water. Examples of viscosity-increasing excipients include high molecular weight polyethylene glycols (e.g., greater than about 5000 daltons), ethyl cellulose, hydroxypropyl cellulose, hydroxypropyl methyl cellulose, methyl cellulose, silicon dioxide, microcrystalline cellulose, magnesium silicate, sugars, and salts.

When the multiparticulates comprise a low-solubility drug and a pore-forming carrier, the multiparticulates may be made by a process comprising the steps of (a) forming a molten mixture comprising drug and the pore-forming carrier, (b) delivering the molten mixture of step (a) to an atomizing means to form droplets from the molten mixture, and (c) congealing the droplets from step (b) to form the multiparticulates.

“Molten mixture” as used herein refers to a mixture of low-solubility drug and pore-forming carrier heated sufficiently that the mixture becomes sufficiently fluid that the mixture may be formed into droplets or atomized. Atomization of the molten mixture may be carried out using any of the atomization methods described below. Generally, the mixture is molten in the sense that it will flow when subjected to one or more forces such as pressure, shear, and centrifugal force, such as that exerted by a centrifugal or spinning-disk atomizer. Thus, the drug/carrier mixture may be considered “molten” when any portion of the mixture becomes sufficiently fluid that the mixture, as a whole, may be atomized. Generally, a mixture is sufficiently fluid for atomization when the viscosity of the molten mixture is less than about 20,000 cp, preferably less than about 15,000 cp, more preferably less than about 10,000 cp, even more preferably less than about 5,000 cp, and most preferably less than about 1,000 cp.

The drug in the molten mixture may be dissolved in the pore-forming carrier, may be a suspension of drug distributed in the molten pore-forming carrier, or any combination of such states or those states that are in between. Preferably the molten mixture is a homogeneous suspension of the drug in the molten carrier where the fraction of the drug that melts or dissolves in the molten carrier is kept relatively low. Preferably less than about 30 wt % of the total drug melts or dissolves in the molten carrier. It is preferred that the drug be present in crystalline form in the molten mixture.

Virtually any process may be used to form the molten mixture. One method involves heating the pore-forming carrier in a tank until it is fluid and then adding the drug to the molten carrier. Generally, the pore-forming carrier is heated to a temperature of about 10° C. or more above the temperature at which it becomes fluid. The process is carried out so that at least a portion of the molten mixture remains fluid until atomized. Once the pore-forming carrier has become fluid, the drug may be added to the fluid carrier or “melt.” Although the term “melt” generally refers specifically to the transition of a crystalline material from its crystalline to its liquid state, which occurs at its melting point, and the term “molten” generally refers to such a crystalline material in its fluid state, as used herein, the terms are used more broadly, referring in the case of “melt” to the heating of any material or mixture of materials sufficiently that it becomes fluid in the sense that it may be pumped or atomized in a manner similar to a crystalline material in the fluid state. Likewise “molten” refers to any material or mixture of materials that is in such a fluid state. Alternatively, the drug and the solid carrier may be added to the tank at the same time and the mixture heated until the carrier has become fluid.

Once the molten mixture has been formed, the multiparticulates are made using the process described herein below.

A key characteristic of multiparticulates of the present invention comprising a low-solubility drug and a pore-forming carrier is that they form a highly porous matrix upon introduction to an aqueous environment, permitting rapid ingress of water into the matrix, as well as rapid egress of drug from the matrix, thus facilitating rapid release of the low-solubility drug in an aqueous environment of use.

The multiparticulates of the invention comprising a low-solubility drug and a pore-forming carrier provide a relatively rapid release of drug starting within a few minutes of introduction to an aqueous environment. Generally, following administration of the multiparticulates to the use environment, at least about 50 wt % of the low-solubility drug in the multiparticulates is released to the use environment within one hour. Preferably, at least 70 wt %, and more preferably at least about 80 wt % of the low-solubility drug in the multiparticulates is released to the use environment within one hour of administration thereto. Such relatively rapid release of the low-solubility drug is in contrast to the relatively slower release of low-solubility drugs from the waxy multiparticulate matrices formed in a typical melt-congeal process.

The amount of drug released from the multiparticulates may be determined by introducing the multiparticulates to an aqueous use environment and determining the concentration of drug dissolved in the use environment over time. Preferably, the aqueous use environment is a phosphate-buffered solution consisting of an aqueous solution of 50 mM KH₂PO₄ and 0.5 wt % of the surfactant polyoxyethylene 80 sorbitan monooleate (commercially available as TWEEN 80 from ICI Americas, Inc. of Wilmington, Del.), at pH 7.3 and 37° C. However, because the drug has a low solubility, this test must be performed under conditions where there is a sufficient quantity of buffer so that all of the drug in the multiparticulates can be dissolved. Alternatively, a sample of the multiparticulates can periodically be removed from the buffer solution and the amount of drug remaining in the multiparticulates measured using standard techniques.

### Spray-Congeal Process

The multiparticulates of the present invention are formed using a spray-congeal process as follows. First, once the feed mixture has been formed, it is mixed to ensure the drug is substantially uniformly distributed therein. Mixing is generally done using mechanical means, such as overhead mixers, magnetically driven mixers and stir bars, planetary mixers, and homogenizers. Optionally, the contents of the tank can be pumped out of the tank and through an in-line, static mixer or extruder and then returned to the tank. The amount of shear used to mix the feed mixture should be sufficiently high to ensure substantially uniform distribution of the drug therein. The feed mixture can be mixed from a few minutes to several hours, the mixing time being dependent on the viscosity of the feed. Generally, it is preferred to limit the mixing time to near the minimum necessary to suspend the low-solubility drug substantially uniformly throughout the aqueous sugar alcohol solution or the molten pore-forming carrier.

Alternatively, the feed mixture may be formed in an extruder. By the term “extruder” is meant a device or collection of devices that creates a molten extrudate by heat and/or shear forces and/or produces a uniformly mixed extrudate from a solid and/or liquid (i.e., molten) feed. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten feed; gear-pump extruders, consisting of a heated gear pump, generally counter-rotating, that simultaneously heats and pumps the molten feed; and conveyer extruders. Conveyer extruders comprise a conveyer means for transporting solid and/or powdered feeds, such as a screw conveyer or pneumatic conveyer, and a pump. At least a portion of the conveyer means is heated to a sufficiently high temperature to produce the feed mixture. The feed mixture may optionally be directed to an accumulation tank, before being directed to a pump, which directs the feed mixture to an atomizer. Optionally, an in-line mixer may be used before or after the pump to ensure the feed mixture is substantially homogeneous. In each of these extruders the feed mixture is mixed to form a uniformly mixed extrudate. Such mixing may be accomplished by various mechanical and processing means, including mixing elements, kneading elements, and shear mixing by backflow. Thus, in such devices, the composition is fed to the extruder, which produces a feed mixture that can be directed to the atomizer.

Once the feed mixture has been formed, it is delivered to an atomizer that breaks it into small droplets. Virtually any method can be used to deliver the feed mixture to the atomizer, including the use of pumps and various types of pneumatic devices such as pressurized vessels or piston pots. Typically, the feed mixture is maintained at an elevated temperature while delivering the mixture to the atomizer to prevent solidification and to keep it flowing.

Generally, atomization occurs in one of several ways, including (1) by “pressure” or single-fluid nozzles; (2) by two-fluid nozzles; (3) by centrifugal or spinning-disk atomizers, (4) by ultrasonic nozzles; or (5) by mechanical vibrating nozzles. Detailed descriptions of atomization processes can be found in Lefebvre, Atomization and Sprays (1989) or in Perry's Chemical Engineers' Handbook (7th Ed. 1997). In a preferred embodiment, the atomizer is a centrifugal or spinning-disk atomizer, such as the FX1 100-mm rotary atomizer manufactured by Niro NS of Soeborg, Denmark.

Once the feed mixture has been atomized, the droplets are congealed, typically by contact with a gas at a temperature below the solidification temperature of the droplets. Typically, it is desirable that the droplets are congealed in less than about 60 seconds, preferably in less than about 10 seconds, more preferably in less than about 1 second. Often, congealing at ambient temperature results in sufficiently rapid solidification of the droplets.

When the multiparticulates comprise a low-solubility drug and a sugar alcohol, the feed mixture contains water at high temperature. During the congealing step the water rapidly evaporates from the droplets once they are formed. This in turn results in evaporative cooling, which helps to rapidly congeal the droplets. Once the multiparticulates have been formed, they are further processed by drying them to remove residual water content, then sieving them. Suitable drying processes include tray drying, vacuum drying, fluid bed drying, microwave drying, belt drying, rotary drying, and other drying processes known in the art. The drying process is preferably conducted at a temperature of about 35° to 40° C.

### Excipients and Dosage Forms

The multiparticulates of the present invention may be incorporated into suitable dosage forms, such as tablets, capsules, suspensions, powders for suspension, creams, transdermal patches, depots, and the like. Compositions containing the multiparticulates of the invention may be delivered by a wide variety of routes, including, but not limited to, oral, nasal, rectal, vaginal, subcutaneous, intravenous and pulmonary. Generally, oral delivery is preferred. Inclusion of excipients with the multiparticulates to form dosage forms may be beneficial, and in some cases preferred. The multiparticulates may be added to other dosage form ingredients in essentially any manner that does not substantially alter the drug's activity. Such excipients are well-known in the art, and are described in Remington: The Science and Practice of Pharmacy (20^{th} Ed. 2000). Generally, dosage form excipients such as matrix materials, fillers, diluents, disintegrating agents, solubilizers, drug-complexing agents, pigments, binders, lubricants, glidants, flavorants, and so forth may be used for customary purposes and in typical amounts without adversely affecting the properties of the multiparticulates.

Examples of matrix materials, fillers, or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, and hydroxypropyl methyl cellulose.

Examples of drug-complexing agents or solubilizers include the polyethylene glycols, caffeine, xanthene, gentisic acid and cyclodextrins.

Examples of disintegrants include sodium starch glycolate, sodium carboxymethyl cellulose, calcium carboxymethyl cellulose, croscarmellose sodium, crospovidone (polyvinylpolypyrrolidone), methylcellulose, microcrystalline cellulose, powdered cellulose, starch, pregelatinized starch, and sodium alginate.

Examples of binders include acacia, alginic acid, carbomer, carboxymethyl cellulose sodium, dextrin, ethylcellulose, gelatin, guar gum, hydrogenated vegetable oil, hydroxyethyl cellulose, hydroxypropyl cellulose, hydroxypropyl methyl cellulose, methyl cellulose, liquid glucose, maltodextrin, polymethacrylates, povidone, pregelatinized starch, sodium alginate, starch, sucrose, tragacanth, and zein.

Examples of lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate.

Examples of glidants include silicon dioxide, talc and cornstarch.

The multiparticulates of the invention may also be used in a wide variety of dosage forms for administration of drugs. Exemplary dosage forms are powders or granules that may be taken orally either dry or reconstituted by addition of water or other liquids to form a paste, slurry, suspension or solution; tablets; capsules; and pills. Various additives may be mixed, ground, or granulated with the multiparticulates of the invention to form a material suitable for forming such dosage forms. The dosage forms may be formulated to release the drug rapidly to the mouth, or formulated to provide an initial delay followed by rapid release at the target location.

The multiparticulates of the invention may be formulated in forms that permit them to be delivered as a suspension of particles in a liquid vehicle. Such suspensions may be formulated as a liquid or paste at the time of manufacture, or they may be formulated as a dry powder with a liquid, typically water, added at a later time but prior to oral administration. Powders that are incorporated into a suspension are often termed a “sachet” or an “oral powder for constitution” (OPC). Such dosage forms can be formulated and reconstituted by any known procedure. The simplest approach is to formulate the dosage form as a dry powder that is reconstituted by simply adding water and agitating. Alternatively, the dosage form may be formulated as a liquid and a dry powder that are combined and agitated to form the oral suspension. In yet another embodiment, the dosage form can be formulated as two powders that are reconstituted by first adding water to one powder to form a solution to which the second powder is combined with agitation to form the suspension.

When formulating the multiparticulates into dosage forms, care must be taken to ensure that the drug is delivered to the target location. Since drug release is rapid upon contact with an aqueous environment of use, dosage forms such as a sachet or an OPC may release the drug rapidly in the liquid vehicle or in the mouth. To prevent unwanted release in the mouth, the multiparticulates may be coated with an anti-enteric polymer that does not dissolve in the higher pH of the mouth, but rapidly dissolves in the relatively low pH of the stomach. Such coatings are well-known in the art. Likewise, the multiparticulates may be incorporated into coated tablets or capsules to ensure the drug is rapidly released to the targeted location.

Generally, it is preferred that the multiparticulates be formulated for long-term storage in the dry state as this promotes the chemical and physical stability of the drug.

Without further elaboration, it is believed that one of ordinary skill in the art can, using the foregoing description, utilize the present invention to its fullest extent. Therefore, the following specific examples are to be construed as merely illustrative and not restrictive of the scope of the invention. Those of ordinary skill in the art will understand that known variations of the conditions and processes of the following examples can be used.

### SCREENING EXAMPLES

Sugar alcohols were screened using the following procedure to determine their suitability in making multiparticulates of the present invention. A sample of the sugar alcohol was placed into a glass vial and heated to above the melting point of the sugar alcohol. Several droplets of the melted sugar alcohol were then placed on a glass slide held at room temperature and the propensity of the sugar alcohol to congeal and crystallize was observed. The results of these screening tests are presented in Table 1 and show that sugar alcohols with a high Tm/Tg (K/K) ratio rapidly crystallize, indicating they would be suitable for forming multiparticulates. The sugar alcohols with Tm/Tg ratios of less than 1.5 supercooled under the conditions tested and did not rapidly crystallize, indicating they would not be suitable for forming multiparticulates.

TABLE 1

| | | Glass-Transition | | Screening Test | |
|---|---|---|---|---|---|
| Sugar | Melting Point | Temperature | Tm/Tg | Temperature | |
| Alcohol | (° C. [K]) | (° C. [K]) | (K/K) | (° C.) | Observations |
| Mannitol | 165 [438] | −39 [234] | 1.87 | 170 | Drops rapidly |
| | | | | | crystallize |
| Erythritol | 121 [394] | −42 [231] | 1.71 | 130 | Drops rapidly |
| | | | | | crystallize |
| Xylitol | 94 [367] | −22 [251] | 1.46 | 100 | Drops |
| | | | | | supercool |
| Sorbitol | 97 [370] | −5 [268] | 1.38 | 100 | Drops |
| | | | | | supercool |
| Maltitol | 150 [423] | 47 [320] | 1.32 | 170 | Drops slowly |
| | | | | | congeal |

### Example 1

Multiparticulates comprising 15.6 wt % of the low-solubility drug valdecoxib and 84.4 wt % of the sugar alcohol mannitol were prepared using the following melt-congeal procedure. First, 270 g of the mannitol and 180 g of deionized water were added to a beaker and heated for about 20 minutes in an oven to form a melt at a temperature of slightly less than 100° C. Next, 70 g of valdecoxib was stirred into the solution and mixed at a speed of 2000 rpm for 5 minutes, resulting in a homogeneous feed suspension of the drug in the solution.

The feed suspension was then pumped by a gear pump at a rate of 92 g/min to the center of a spinning-disk atomizer. The spinning disk atomizer was custom-made, consisting of a bowl-shaped stainless steel disk of 10.1 cm (4 inches) in diameter. The surface of the disk was heated with a thin film heater beneath the disk to about 100° C. The disk was mounted on a motor that rotated the disk at a speed of approximately 10,000 RPM. The entire assembly was enclosed in a plastic bag approximately 8 feet in diameter to allow congealing and to capture multiparticulates formed by the atomizer. Ambient air was introduced from a port underneath the disk to provide cooling of the multiparticulates upon congealing and to inflate the bag to its extended size and shape.

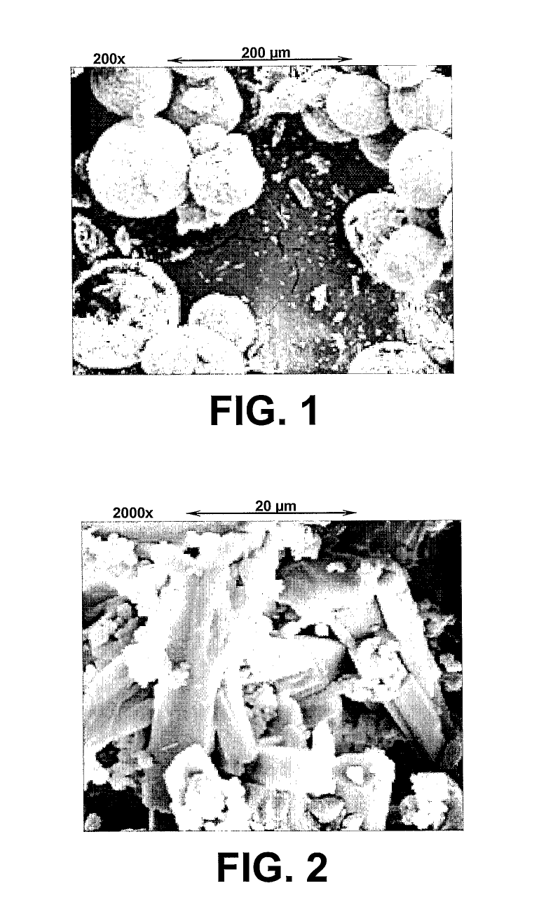

The particles formed by the spinning-disk atomizer were congealed in the ambient air and a total of 263 g of multiparticulates were collected and tray-dried for 2½ days at 38° C., resulting in 210 g of dry multiparticulates. The mean particle size was about 100 μm based on analysis by Scanning Electron Microscope (SEM). FIG. 1 shows an SEM image of a sample of these multiparticulates at 200×. The internal structure of the so-formed multiparticulates is shown in another SEM photo at 2000× (FIG. 2) and confirms the distribution of drug crystals throughout a matrix of larger rod-shaped mannitol crystals.

The rate of disintegration of these multiparticulates was determined using the following procedure. A 47 mg sample of the multiparticulates was placed into a USP Type 2 dissoette dissolution flask equipped with Teflon-coated paddles rotating at 100 rpm. The dissolution flask contained 900 mL of a phosphate-buffered solution comprising an aqueous solution of 50 mM KH₂PO₄ and 0.5 wt % polyoxyethylene 20 oleate, pH 7.3, held at 37.0±0.5° C. Samples were taken using a syringe attached to a cannula with a 70 μm filter. A sample of the fluid in the flask was drawn into the syringe, the cannula was removed, and a 0.45-μm filter was attached to the syringe. One mL of sample was filtered into a High Performance Liquid Chromatography (HPLC) vial. Samples were collected at 1, 3, 5 and 10 minutes following addition of the multiparticulates to the flask. The samples were analyzed using HPLC (Zorbax SB-C8 column, 3.5 μm particles, 7.5 cm×4.6 mm i.d.; mobile phase of 55/45 5 mM triethanolamine, pH 7.0/acetonitrile at 1.5 mL/min; UV absorbance measured at 256 nm with a diode array spectrophotometer).

The amount of drug released was calculated based on the potency assay of the formulation. To assay the potency of the multiparticulates, about 17 mg of the multiparticulates were weighed and added to a 25 mL volumetric flask. Next, about 10 mL acetonitrile/methanol (80/20 vol/vol) was added, and the solution was sonicated for 15 minutes. The flask was cooled to room temperature and filled to volume with acetonitrile/methanol (80/20 vol/vol). An aliquot of the solution was then centrifuged for 5 minutes at 13,000 rpm, and analyzed to determine the total amount of drug in the formulation. The potency assay of the formulation was used to calculate the amount of drug added for each dissolution test. The amount of drug in each sample was divided by the total amount of drug added for the test, and the results are reported as percent of assay. The results of these dissolution tests are given in Table 2, which shows 80 wt % of the valdecoxib was released in less than 2 minutes.

TABLE 2

| Time | Valdecoxib Released |
|---|---|
| (min) | (wt %) |
| 0 | 0 |
| 1 | 75 |
| 2 | 95 |
| 3 | 102 |
| 5 | 104 |
| 10 | 103 |

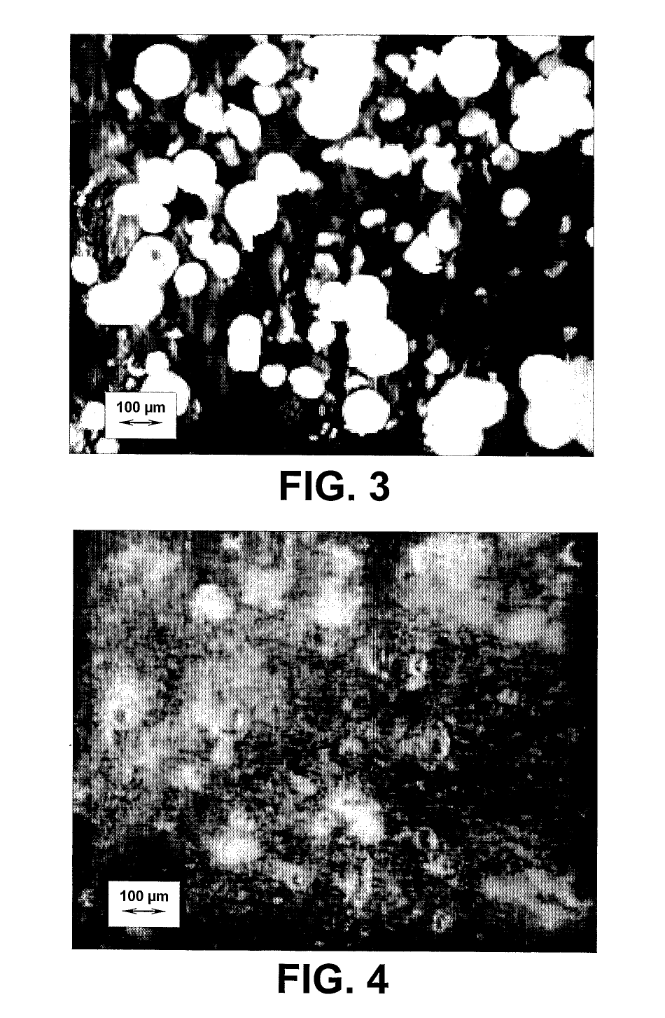

Samples of the multiparticulates were also examined under a light microscope before and after exposure to deionized water. FIG. 3 shows a photograph of the multiparticulates of Example 1 before exposure to water, while FIG. 4 shows a photograph of the multiparticulates of Example 1 about 5 seconds after exposure to deionized water. These images demonstrate rapid disintegration of the multiparticulates when exposed to water.

### Example 2

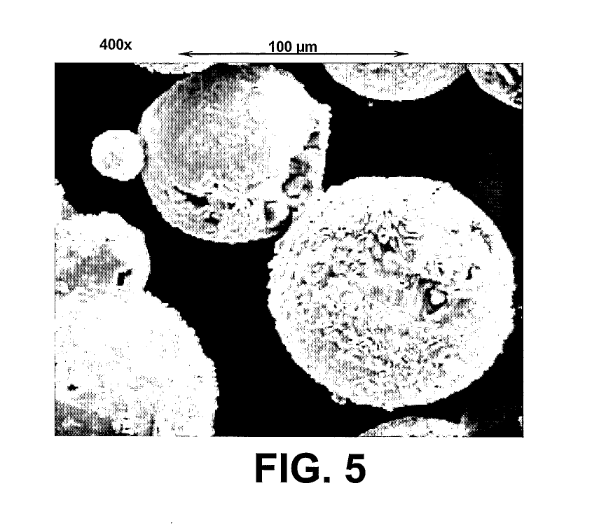

Multiparticulates comprising 29.4 wt % of the low solubility drug ziprasidone hydrochloride and 70.6 wt % mannitol were made as in Example 1 with the following exceptions. The aqueous mannitol solution consisted of 60/40 (w/w) mannitol/water at 100° C. The feed mixture consisted of 20 wt % ziprasidone hydrochloride, 48 wt % mannitol, and 32 wt % deionized water. The feed mixture was stirred at 700 rpm at 100° C. for 5 minutes before being pumped to the spinning-disk atomizer at a rate of 75 g/min. The disk speed was 10,000 rpm. The so-formed multiparticulates were collected and try-dried overnight at 38° C. The mean particle size was about 120 μm, as determined by SEM analysis. FIG. 5 shows an SEM image of a sample of these multiparticulates at 400×.

The rate of disintegration of these multiparticulates was determined as in Example 1 with the following exceptions. About 313 mg of multiparticulates were added to a buffer solution consisting of 0.05 M NaH₂PO₄, at pH 6.5, containing 2 wt % sodium lauryl sulfate (SLS). Samples were collected at the times indicated in Table 3. Samples were analyzed using HPLC (Zorbax Rx C8 Reliance, 4.0×80 mm column, with a mobile phase of 55/45 50 mM KH₂PO₄, pH 6.5/acetonitrile, at 1.0 mL/min, and UV absorbance measured at 315 nm with a diode array spectrophotometer). The results are given in Table 3, which shows that 80 wt % of the drug was released within 3 minutes.

TABLE 3

| | Ziprasidone |
|---|---|
| Time | Released |
| (min) | (wt %) |
| 0 | 0 |
| 3 | 80 |
| 5 | 80 |
| 10 | 82 |
| 15 | 85 |
| 20 | 87 |
| 30 | 91 |
| 45 | 94 |
| 60 | 96 |
| 90 | 98 |
| 120 | 99 |

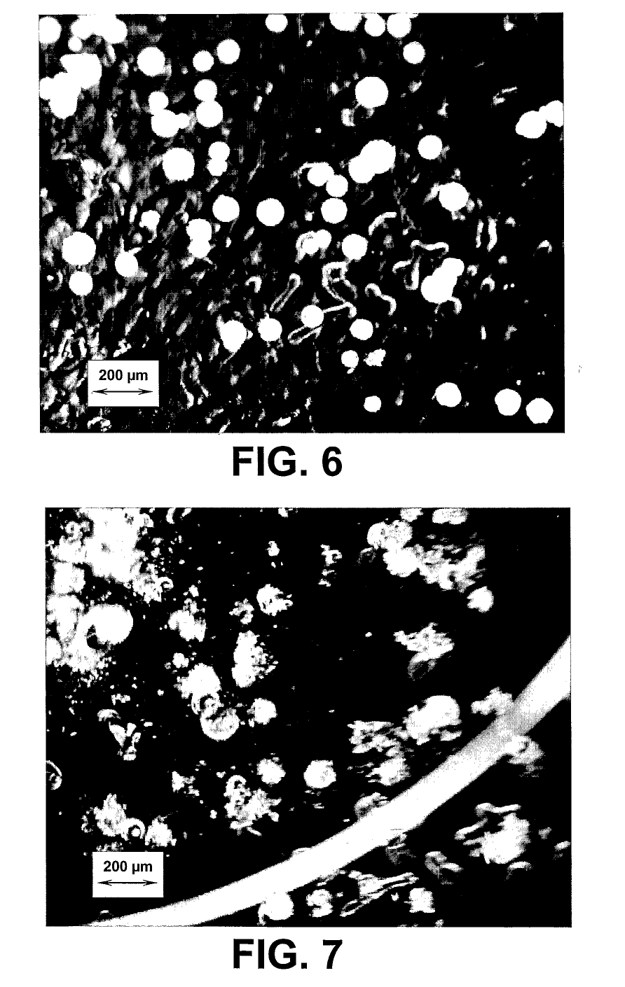

Samples of the so formed multiparticulates were also examined under a light microscope before and after exposure to water. FIG. 6 shows a photograph of the multiparticulates before exposure to water, while FIG. 7 shows a photograph of the same multiparticulates about 5 seconds after exposure to deionized water. These images demonstrate rapid disintegration of the multiparticulates upon exposure to water.

### Example 3

Multiparticulates comprising 12.9 wt % of the low-solubility drug valdecoxib and 87.1 wt % erythritol were made as in Example 1 with the following exceptions. The aqueous erythritol solution consisted of 75/25 (w/w) erythritol/water at 90° C. The feed mixture consisted of 10 wt % valdecoxib, 67.5 wt % erythritol, and 22.5 wt % deionized water. The feed homogenized at 4500 rpm for 5 minutes before being pumped to the spinning-disk atomizer at a rate of 150 g/min. The disk speed was 10,000 rpm. The so-formed multiparticulates were collected and try-dried overnight at 40° C. The volume mean particle size was about 270 μm, as determined using a Malvern particle-size analyzer.

The rate of disintegration of these multiparticulates was determined as in Example 1 and showed that more than 80 wt % of the valdecoxib was released within 1 minute following administration to the aqueous environment of use.

### Examples 4-6

Multiparticulates of the low-solubility drug valdecoxib, a pore-forming carrier and a dissolution enhancer were prepared using the following melt-congeal procedure. First, the pore-forming carrier and the dissolution enhancer were added to a beaker and heated for about 20 minutes in an oven to form a melt at a temperature of about 90° C. Next, the drug was added to the melt and stirred at 700 rpm for 5 minutes, resulting in a homogeneous feed suspension of the drug in the molten mixture.

The feed suspension was then pumped by a syringe pump at a rate of 75 g/min to the center of a centrifugal atomizer, described in Example 1. The surface of the disk was heated to about 90° C. The disk speed was approximately 10,000 RPM. The multiparticulates formed by the centrifugal atomizer were congealed in the ambient air.

The multiparticulates of Example 4 consisted of 30 wt % valdecoxib, 60 wt % of the pore-forming carrier COMPTRITOL HD5, and 10 wt % of the dissolution enhancer GELUCIRE 50/13. The so-formed multiparticulates had a mean particle size estimated to be about 80 μm as determined by SEM analysis. For Example 5, the multiparticulates consisted of 30 wt % valdecoxib, 40 wt % COMPRITOL HD5 and 30 wt % GELUCIRE 50/13, and had a mean particle diameter of about 90 μm. For Example 6, the multiparticulates consisted of 30 wt % valdecoxib, 40 wt % COMPRITOL HD5, and 25 wt % GELUCIRE 50/13 and 5 wt % polysorbate 80 as dissolution enhancers, and had a mean particle diameter of about 90 μm.

The rate of drug release from these multiparticulates was determined using the procedures described in Example 1. The results of these dissolution tests are given in Table 4.

Table 4

| | | Valdecoxib |
|---|---|---|
| | Time | Released |
| Example No. | (min) | (wt % assay) |
| 4 | 0 | 0 |
| | 1 | 3 |
| | 2 | 5 |
| | 3 | 6 |
| | 5 | 9 |
| | 10 | 15 |
| | 20 | 26 |
| | 30 | 35 |
| | 60 | 56 |
| 5 | 0 | 0 |
| | 1 | 6 |
| | 2 | 10 |
| | 3 | 13 |
| | 5 | 19 |
| | 10 | 29 |
| | 20 | 44 |
| | 30 | 55 |
| | 60 | 76 |
| 6 | 0 | 0 |
| | 1 | 7 |
| | 2 | 13 |
| | 3 | 16 |
| | 5 | 23 |
| | 10 | 35 |
| | 20 | 52 |
| | 30 | 64 |
| | 60 | 85 |

The results in Table 4 show that the multiparticulates of Example 4 released 56 wt % of the drug after one hour. The multiparticulates of Example 5 released 76 wt % of the drug after one hour, while the multiparticulates of Example 6 released 85 wt % of the drug after one hour.

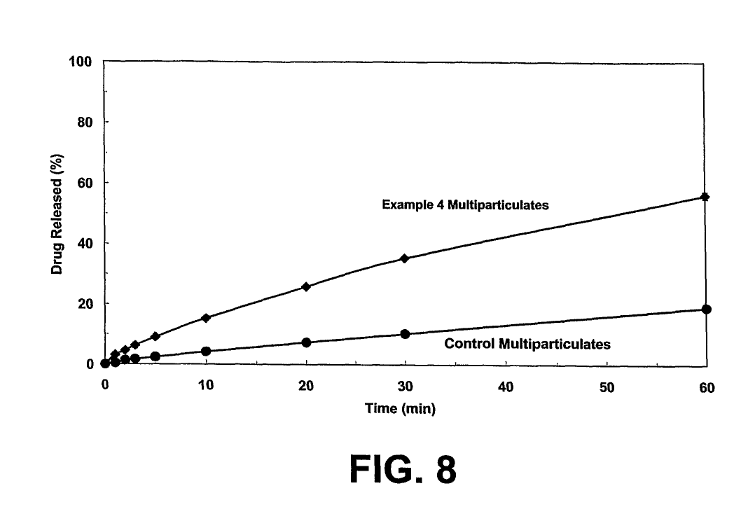

As a control, the same test was performed with multiparticulates formed in the same fashion consisting of 20 wt % valdecoxib, 70 wt % of a more hydrophobic carrier consisting of a mixture of mono-, di- and glycercyl tribehenates (COMPRITOL 888 ATO from Gattefosse) and 10 wt % GELUCIRE 50/13 as a dissolution enhancer. The results are shown in FIG. 8, together with those for Example 4, and show that only about 20 wt % of the drug was released from the control within 60 minutes, as compared to 56 wt % for the multiparticulates of Example 4 within the same time period.

### Examples 7-8

Multiparticulates were made as in Example 4 with the following exceptions. The multiparticulates comprised valdecoxib and a pore-forming carrier comprising a mixture of solid aliphatic alcohols consisting of an aqueous solution of stearyl and cetyl alcohols with a minor amount of the surfactant sodium lauryl sulfate (LANETTE SX). The multiparticulates of Example 7 comprised 20 wt % valdecoxib and 80 wt % LANETTE SX. The multiparticulates of Example 8 comprised 20 wt % valdecoxib, 75 wt % LANETTE SX, and 5 wt % TWEEN 80. The disk temperature was set at 75° C. when making the multiparticulates of Example 7 and set at 90° C. when making the multiparticulates of Example 8. The mean diameter for the multiparticulates of Example 7 was about 120 μm, while the mean diameter for the multiparticulates of Example 8 was about 90 μm.

The rate of release of valdecoxib from the multiparticulates of Examples 7-8 was determined as in Example 4. The results of these dissolution tests are given in Table 5, and show that the multiparticulates of Example 7 released 60 wt % of the drug within one hour, while the multiparticulates of Example 8 released 98 wt % of the drug within one hour.

TABLE 5

| | | Valdecoxib |
|---|---|---|
| | Time | Released |
| Example No. | (min) | (% assay) |
| 7 | 0 | 0 |
| | 1 | 1 |
| | 2 | 2 |
| | 3 | 4 |
| | 5 | 6 |
| | 10 | 14 |
| | 20 | 26 |
| | 30 | 37 |
| | 60 | 60 |
| 8 | 0 | 0 |
| | 1 | 14 |
| | 2 | 22 |
| | 3 | 30 |
| | 5 | 46 |
| | 10 | 56 |
| | 20 | 74 |
| | 30 | 84 |
| | 60 | 98 |

### Examples 9-10

Multiparticulates were made as in Example 4 with LANETTE SX and the low-solubility drug ziprasidone hydrochloride. The multiparticulates of Example 9 comprised 20 wt % drug and 80 wt % LANETTE SX. The multiparticulates of Example 10 comprised 50 wt % drug and 50 wt % LANETTE SX. The multiparticulates of Examples 9 and 10 had mean diameters of about 80 μm based on SEM analysis.

The rate of drug release from the multiparticulates of Examples 9-10 was determined as in Example 4, except that the dissolution test solution consisted of 900 mL of 50 mM NaH₂PO₄ buffer, pH 6.5, with 2 wt % sodium lauryl sulfate at 37.0±0.5° C., and the following HPLC procedure was used. Samples were analyzed using a Zorbax Rx C8 Reliance, 4.0×80 mm column, with a mobile phase of 55/45 50 mM KH₂PO₄, pH 6.5/acetonitrile, a flow rate of 1.0 mL/min, and UV absorbance was measured at 315 nm with a diode array spectrophotometer. The results of these dissolution tests are given in Table 6, and show rapid and virtually complete drug release within an hour.

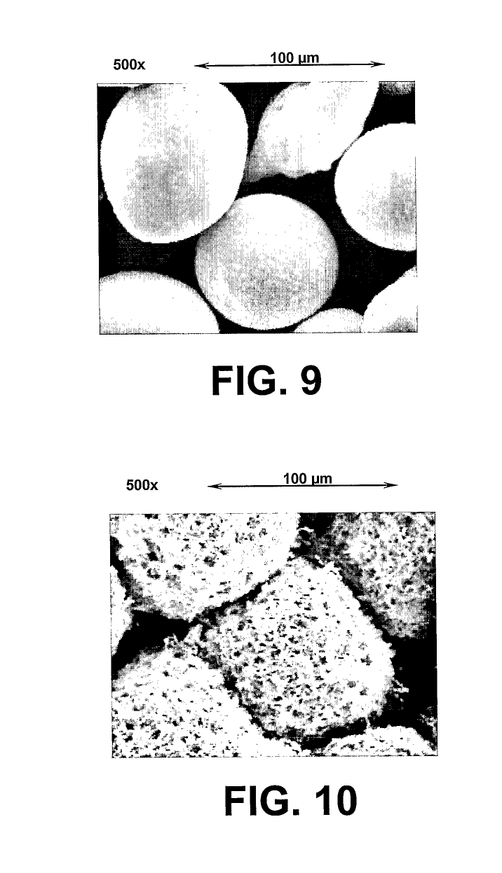

SEM photos of the multiparticulates of Example 9 were taken, and they are shown at 500× in FIG. 9. The same batch of multiparticulates shown in FIG. 9 was then exposed to deionized water for short time intervals and the multiparticulates recovered and lyophilized. The lyophilized samples were then examined by SEM. FIG. 10 is an SEM photo of the lyophilized multiparticulates at 500× taken after 60 seconds of contact with the water, and demonstrates the formation of a highly porous matrix in the multiparticulates upon exposure to an aqueous environment.

TABLE 6

| | | Ziprasidone |
|---|---|---|
| | | Hydrochloride |
| | Time | Released |
| Example No. | (min) | (% assay) |
| 9 | 0 | 0 |
| | 3 | 4 |
| | 5 | 11 |
| | 10 | 25 |
| | 15 | 38 |
| | 20 | 50 |
| | 30 | 67 |
| | 45 | 83 |
| | 60 | 91 |
| 10 | 0 | 0 |
| | 3 | 3 |
| | 5 | 9 |
| | 10 | 24 |
| | 15 | 41 |
| | 20 | 56 |
| | 30 | 78 |
| | 45 | 94 |
| | 60 | 98 |

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1-27. (canceled)

28. A process for preparing a pharmaceutical composition comprising the steps:
(a) forming a feed mixture comprising a drug having an aqueous solubility of less than about 1 mg/mL at pH 6-7, a sugar alcohol selected from the group consisting of mannitol, erythritol, and mixtures thereof, and water;
(b) forming droplets of said feed mixture; and
(c) solidifying said droplets to form multiparticulates, wherein said multiparticulates have a volume-weighted mean diameter of from about 10 μm to about 1000 μm.

29. The process of claim 28 wherein step (b) is conducted by a spinning disk atomizer.

30. The process of claim 28 wherein said feed mixture of step (a) is formed by adding drug to an aqueous solution comprising said sugar alcohol and water.

31. The process of claim 30 wherein said aqueous solution has a temperature of less than about 120° C.

32. The process of claim 30 wherein said sugar alcohol is present in said aqueous solution in an amount of at least about 40 wt %.
